(19) United States
(12) Patent Application Publication
(10) Pub. No.: US 2004/0181180 A1
(43) Pub. Date: Sep. 16, 2004

(54) Wood airway, neck, and head support

(76) Inventor: Thomas Oval Wood, Memphis, TN (US)

Correspondence Address: Thomas O. Wood, M.D., 4264 Nellwood Lane, Memphis, TN, 38117, US

(21) Appl. No.: 10/786,211

(22) Filed: Feb. 24, 2004

Related U.S. Application Data
(60) Provisional application No. 60/455,385, filed on Mar. 14, 2003.

(51) Int. Cl. A61F 5/00

(52) U.S. Cl. 602/18

(57) ABSTRACT

An airway, neck, and head support device that assists in stabilizing the airway of a supine patient, given local anesthesia or monitored anesthesia care, by keeping the chin elevated thereby preventing secondary collapse of the airway, and by maintaining head stability for the continuance of uninterrupted, safe surgery. The support device, made of polyurethane foam, can also be used to receive and support the neck and the back of the head prior to surgery thereby providing physical comfort for the patient. Construction of this device was designed to enable the user to rotate it from the back of the head to its selected position between the chin and chest or to place it in various selected positions behind the head to push the neck forward, open the airway, and stabilize the head during local anesthesia or monitored anesthesia care.

### CROSS-REFERENCE TO RELATED APPLICATIONS

This application claims the benefit of provisional patent application Serial No. 60/455,385 filed on Mar. 14, 2003.

### BACKGROUND OF THE INVENTION

1. Field of the Invention

One of the most frequent problems during local anesthesia or monitored anesthesia care is airway obstruction which occurs when a supine patient's chin drops toward the chest and neck. This necessitates manual elevation of the patient's chin and sometimes insertion of an oral or nasal airway. When the chin requires manual elevation to keep the airway open, it becomes difficult to hold the patient in a still position so that surgery can continue safely. When an oral or nasal airway is required to stabilize an airway, surgery must be stopped while the device is inserted thus creating additional risks during surgery.

Furthermore, an obstructed airway stimulates a patient to attempt to reopen the airway causing, for example, the head to suddenly move. A person given local anesthesia or monitored anesthesia care is disadvantaged in that the anesthesia induces sleep as well as general muscle relaxation. Therefore, as the functioning of the muscular support system of the patient is compromised, attempts to reopen an obstructed airway due to the chin dropping toward the chest and neck may manifest in a sudden jerking movement of the head. Patient movement is of concern particularly during a microscopic procedure, such as ophthalmic surgery, because it may result in injury to the patient and possibly distract the surgeon.

It is also desirable for the patient to be as comfortable, as possible, prior to any type of surgery or medical procedure. While in a conscious state and waiting for anesthesia, a patient may feel tense, anxious, or physically uncomfortable. Often, a nurse or other caregiver uses pillows to aid in a patient's comfort. However, at the neck and head area in particular, pillows tend to slide out of position if the patient moves or tend to place the patient's head in an awkward position. Therefore, by contributing to both patient safety and physical comfort, a support device that addresses these needs could benefit the patient.

2. Description of Related Art

The airway, neck, and head of a patient receiving local anesthesia or monitored anesthesia care must be stabilized to ensure that the patient can maintain consistent breathing without the need for constant adjustment. A device that keeps the neck and chin elevated and the head still is desirable because it prevents the airway from collapsing toward the chest and neck and stabilizes the neck and head for safe surgery.

The related art has addressed this problem with various techniques. For example, an anesthetist, anesthesiologist, or nurse will keep the chin elevated and the head still by manually lifting the chin. This can be a tedious and tiring task if the surgery is delicate or lengthy. Another technique requires positioning pillows or towels around the patient's neck and head to keep the airway open and stable. However, rolled-up pillows or towels can slide out of position or place the patient's airway, neck, and head in awkward positions. Needless to say, both techniques do not provide an acceptable means for maintaining airway, neck, and head stability.

The related art has also addressed this problem with various devices. For example, inflatable devices and devices using tethers or other attachments to keep the airway stabilized have been disclosed in U.S. Pat. No. 6,510,574 and U.S. Pat. No. 5,632,283, respectively. Additionally, the related art has presented devices which have numerous parts, such as in U.S. Pat. No. 5,184,365, and appear to be complicated to use. Multi-use support systems encased in protective coverings, such as in U.S. Pat. No. 6,625,831, have also been known in the related art. These require thorough cleaning between uses, are subject to tears in the protective covering, and are often expensive to produce.

The present invention, however, proposes to establish airway, neck, and head stability with a support device that is created with one piece of polyurethane foam, cut to a selected shape, and that is equipped with two adhesive strips and a flexible strap, which further secure it in one of several chosen positions. These include, but are not limited to, securing the device between the chin and chest of the supine patient and positioning the device in selected ways behind the neck and head. The present invention, designed for one use, also affords ease of use and is inexpensive.

Unseen in the related art is an airway, neck, and head support device which can also be used as a pillow prior to surgery. The present invention proposes to meet this need and then serve as an airway, neck, and head stabilizer during local anesthesia or monitored anesthesia care. The present invention proposes various positioning techniques to stabilize the airway, neck, and head of a patient based on its unique contour and design.

### SUMMARY OF THE INVENTION

The present invention was developed to stabilize the airway of a supine patient, receiving local anesthesia or monitored anesthesia care, by keeping the chin elevated, either by securing it between the patient's chin and chest or by positioning it behind the patient's neck and head. After initial sedation, this prevents secondary collapse of the airway and maintains head stability, allowing safe surgery to continue uninterrupted. Additionally, the airway stability established by the present invention enables the patient to breathe consistently, without obstruction and difficulty.

The neck and head stability established by the present invention alleviates the need for the anesthetist, anesthesiologist, or nurse to manually position and hold the patient's head during a local or monitored anesthesia care procedure, particularly surgery. As mentioned, manual manipulation of the chin and head can be difficult and tiring when the procedure is either delicate or lengthy.

With the above advantages being recognized, the airway, neck, and head stability established by the present invention decreases the time that the patient is exposed to anesthesia as well as the duration of surgery. It also decreases the risks associated with inserting an oral or nasal airway during the surgical procedure. Additionally, the present invention enables the surgeon to avoid discontinuing the procedure to position or readjust the patient's head thereby allowing the surgeon to remain focused on the procedure.

Alternatively, the present invention is contoured in a selected manner and constructed of a soft, foam material so that neck and head support, for comfort, can be maintained prior to surgery while the reclining or supine patient is still conscious. The device is placed behind the patient's neck and head so that the backs of the neck and head are supported comfortably. Physical comfort for a patient prior to surgery is important in that it can help reduce anxiety or apprehension associated with surgery.

Because of the chosen contour and construction of this support device, it can then be rotated around the neck and secured in a selected position between the chin and chest or remain behind the patient's head in one of several recommended positions. A flexible, continuous strap can be wrapped around the support device and secured with a tab to maintain proper positioning of the device. To account for the patient's head size and length of the neck, the tightness of the strap is adjustable. The object, then, of the support device is to elevate the chin, thereby keeping the airway open, and to stabilize the head and neck during local anesthesia or monitored anesthesia care, particularly microscopic, ophthalmic surgery.

### BRIEF DESCRIPTION OF THE DRAWINGS

FIG. 1 is a three dimensional view of the support device showing its general contour and design.

FIG. 2 is a top view of the support device.

FIGS. 3 and 4 are top and bottom views of the support device with labels “CHIN” and “CHEST,” respectively.

FIG. 5A is a top view of the strap and its tab.

FIG. 5B is a three dimensional view of the support device showing the top surface with the strap wrapped around it.

FIG. 6A is a right side view of the support device in a placement called pillow position.

FIG. 6B is a left side view of the support device in pillow position.

FIG. 6C is a view, from the top of the head, of the support device in pillow position.

FIG. 7A is a right side view of the support device stabilizing the airway, neck, and head of a patient in a placement between the chin and chest.

FIG. 7B is a left side view of the support device stabilizing the airway, neck, and head of a patient from between the chin and chest.

FIG. 7C is a frontal view of the support device stabilizing the airway, neck, and head of a patient from between the chin and chest.

FIG. 8A is a right side view of the support device stabilizing the airway, neck, and head of a patient in a placement behind the head.

FIG. 8B is a left side view of the support device stabilizing the airway, neck, and head of a patient from behind the head.

FIG. 8C is a view, from the top of the head, of the support device stabilizing the airway, neck, and head of a patient from behind the head.

FIGS. 9A and B are side views of the support device stabilizing the airway, neck, and head of a patient from the second proposed placement behind the head.

FIG. 10A is a left side view of the support device stabilizing the airway, neck, and head of a patient from the third proposed placement behind the head.

FIG. 10B is a frontal view of the support device stabilizing the airway, neck, and head of a patient from the third proposed placement behind the head.

FIG. 10C is a view, from the top of the head, of the support device stabilizing the airway, neck, and head of a patient from the third proposed placement from behind the head.

### DESCRIPTION OF THE PREFERRED EMBODIMENT

The following description sets forth a general representation of the airway, neck, and head support device as established by the inventor. It is provided to further demonstrate the various uses of the present invention.

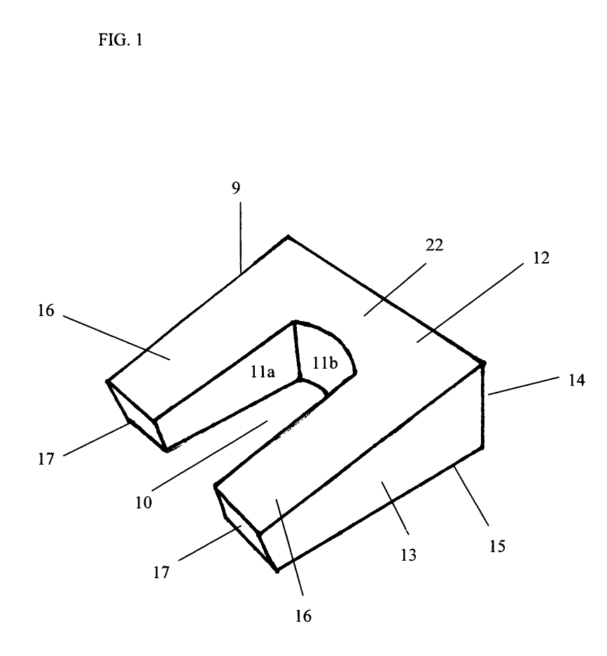

FIG. 1 presents the preferred embodiment of the device generally shown as 9 for supporting and stabilizing the airway, neck, and head of a supine patient, given local anesthesia or monitored anesthesia care. This three dimensional view discloses the general contour and basic design principles of the present invention, as selected by the inventor, but does not restrict the-present invention from variations in its use, contour, or design principles.

In this initial view of support device 9, the carved out central section creating an arch shape, known as the neck trough 10, and one interior side 11a of the neck trough 10 are disclosed. The other interior side, which is not visible, is the mirror image of side 11a. Interior sides 11a are adjoined at the top of neck trough 10 by a curved central portion known as 11b. The top exterior surface generally shown as 12 is disclosed as is one exterior side wall 13. The other exterior side wall, which is not visible, is the mirror image of side 13. Furthermore, the central portion 22, of top exterior surface generally shown as 12, is the section of the support device on which the head, neck, or chin rests for several selected positions.

According to FIG. 1, the preferred embodiment of the device 9 also discloses its general contour. The thicker end of the support device is generally shown as 14 and is created due to the selected angular design of the device 9. Surrounding the neck trough 10 are the legs of the arch 16 and the bases of the arch 17. Furthermore, the bottom exterior surface, on which the device is placed and is, therefore, unseen, is generally represented by 15.

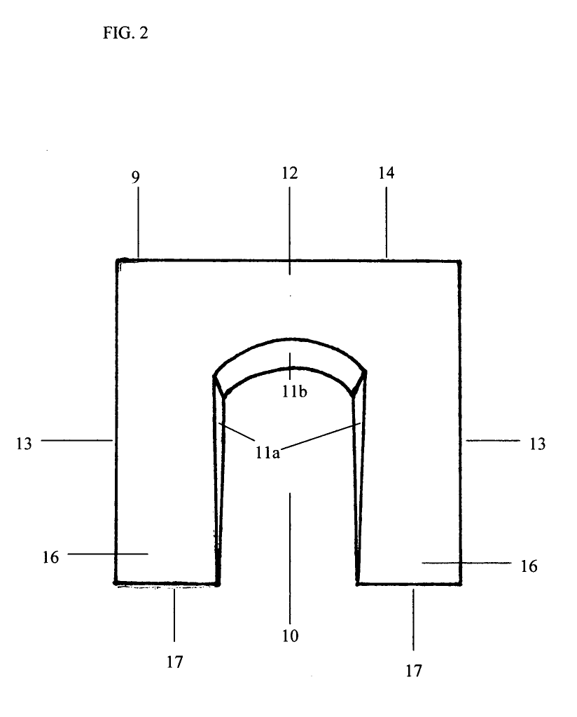

FIG. 2 represents a top view of the airway, neck, and head support device. Again, the carved out central portion is shown as it results in an arch shaped space creating the neck trough 10. The interior sides 11a and 11b are shown as are the legs of the arch 16 and the bases of each leg of the arch 17. The general design also shows that the width of sections 16 and of the space created by 10 is equal to the width of the opposing end 14. This top view further demonstrates how the neck trough 10 is surrounded by one continuous piece of the foam concluding at exterior sides 13, 14, and 17.

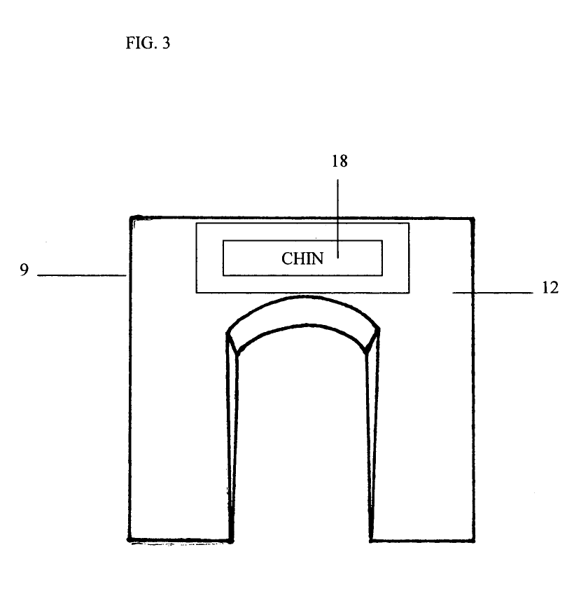

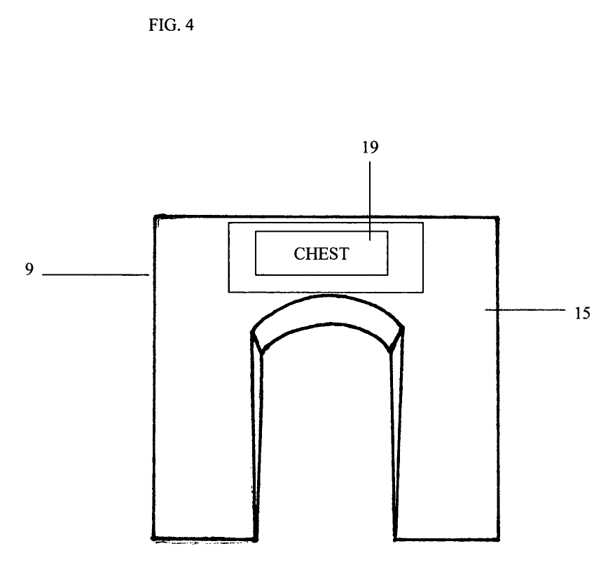

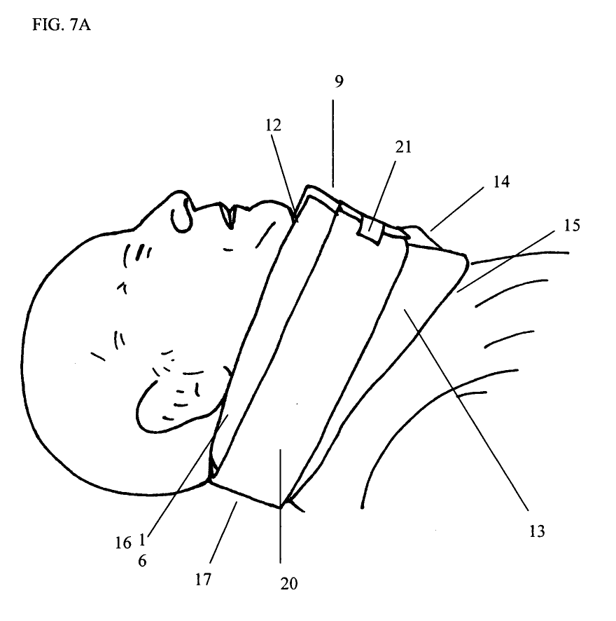

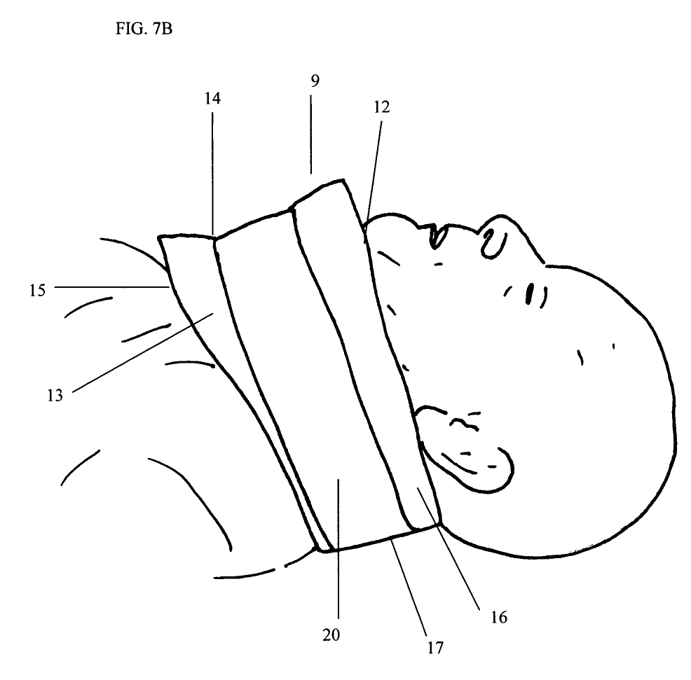

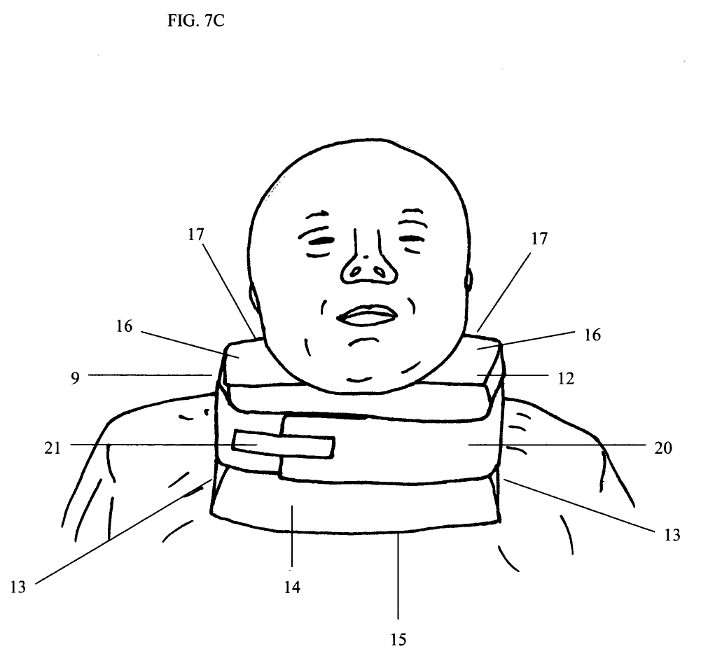

FIG. 3 illustrates another top view of the device 9, as shown in FIG. 2. Identified in FIG. 3 is the top surface 12 with its removable label marked “CHIN” 18. Label 18 covers an adhesive strip which helps to secure the device in its position between the chin and chest, shown in FIGS. 7A-C. FIG. 4 illustrates the corresponding bottom surface 15 with its removable label marked “CHEST” 19. Label 19 covers an adhesive strip which also helps to secure the device in its position between the chin and chest, shown in FIGS. 7A-C.

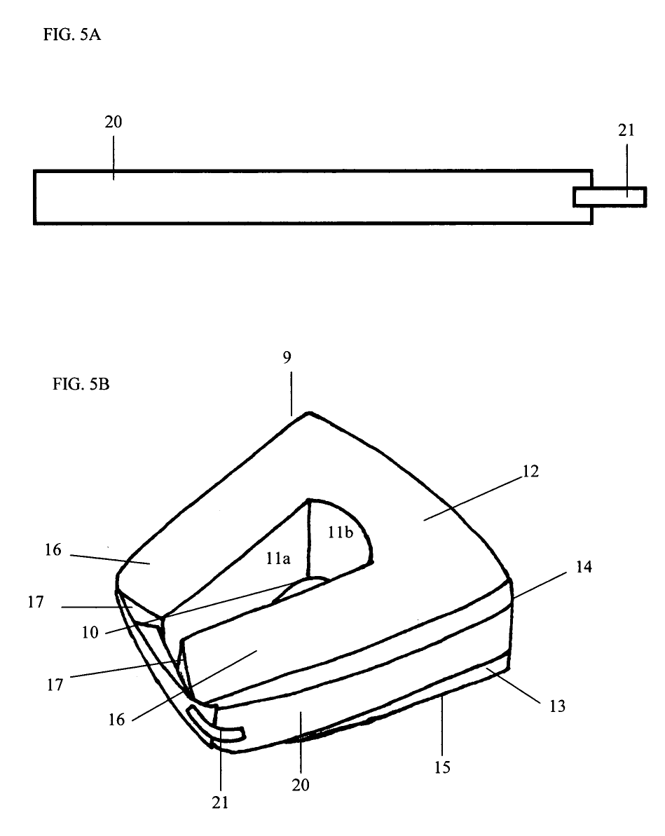

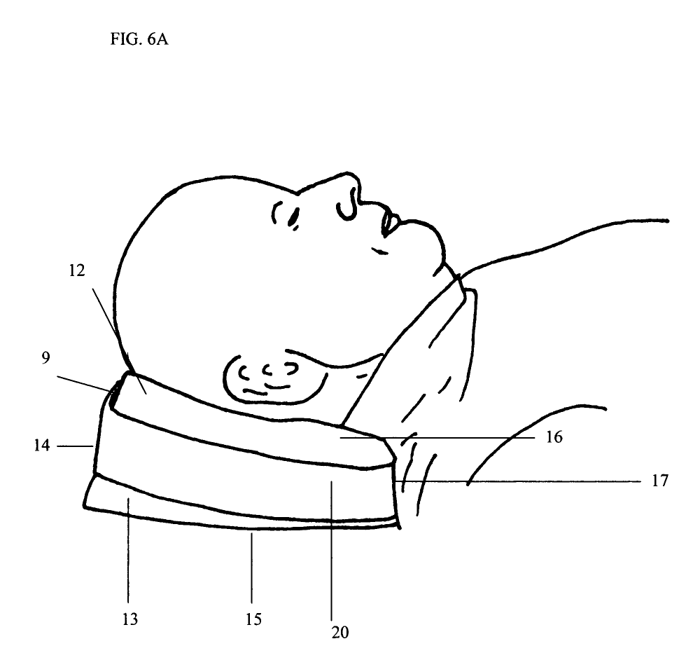

FIG. 5A shows the flexible strap 20 and its tab 21 that is used with the support device. FIG. 5B illustrates another top view of the device 9, as shown in FIG. 1; identifies strap 20 fastened around the support device 9 with tab 21; and shows exterior surface 12 facing up and exterior surface 15 resting on the bed. The strap 20, when wrapped around the entire device, touches exterior surfaces 13 and 14 and crosses exterior surface 17 and the space created by the neck trough 10.

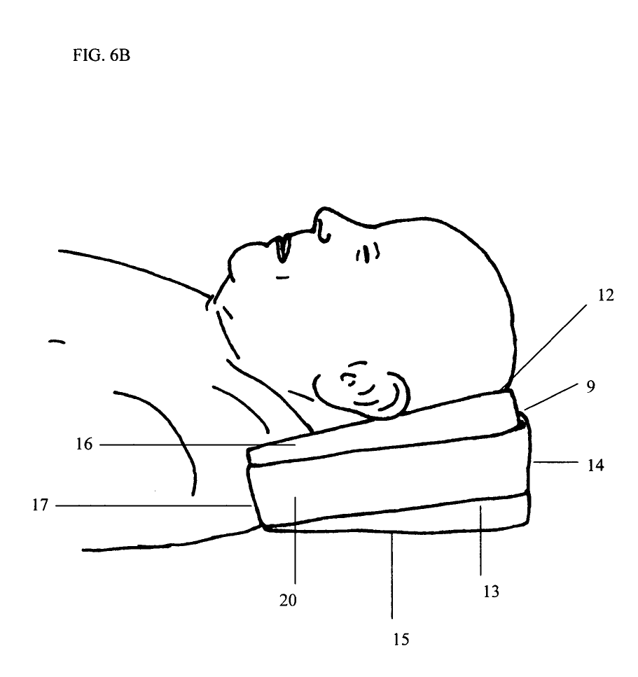

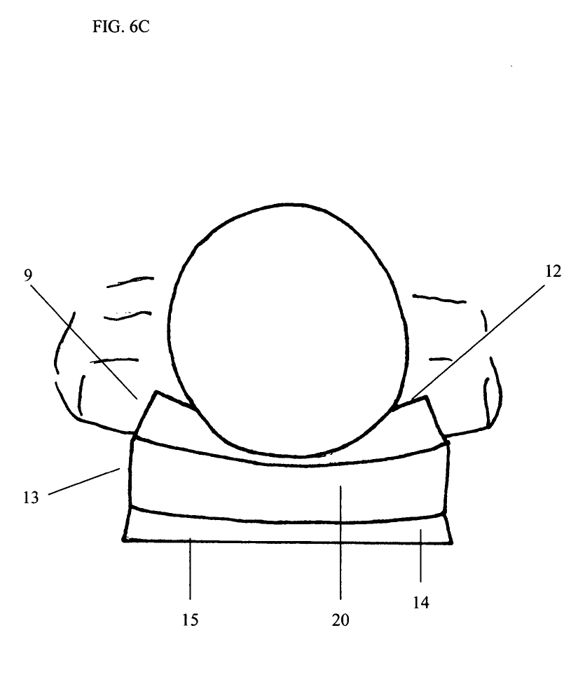

FIG. 6A is a right side view of the support device 9 in pillow position. For this position, the strap 20 is placed around the entire device 9 and fastened with tab 21, as demonstrated by FIG. 5B. The device 9 is then placed behind the patient's neck and head so that the backs of the neck and head are supported comfortably. The general orientation of the device 9 in pillow position, prior to surgery, requires that device 9 be placed behind the head of the patient With the back of the patient's head resting at the thicker end 14 and on the area generally shown as 22, FIG. 1, on top exterior surface 12. The neck trough 10, interior sides 11a and 11b, all shown in FIG. 2, and the legs of the arch 16 correspondingly support the back and sides of the neck. The bases of the arch 17 point toward the patient's shoulders. The exterior bottom surface 15 rests on the bed. Labels 18, FIG. 3, and 19, FIG. 4, remain on the device while it is in pillow position.

FIGS. 6B and C are additional views of the device 9 placed in pillow position. FIG. 6B shows the device 9 in pillow position from the left side. FIG. 6C shows the device 9 from the top of the head.

Upon delivery of anesthesia, the device 9 is then converted from pillow position to one of several positions that help keep the airway open, support the neck, and stabilize the head for surgery. This conversion results from a change in position of device 9.

The first preferred embodiment proposes a method of stabilizing the airway, neck, and head in a position between the chin and chest, as described by FIGS. 7A-C. The second preferred embodiment proposes three methods of stabilizing the airway, neck, and head from behind the head.

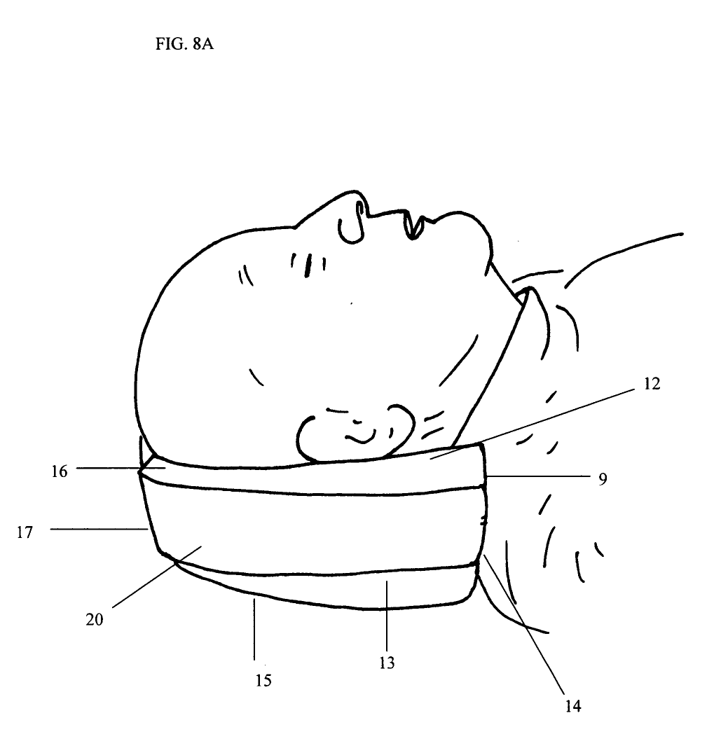

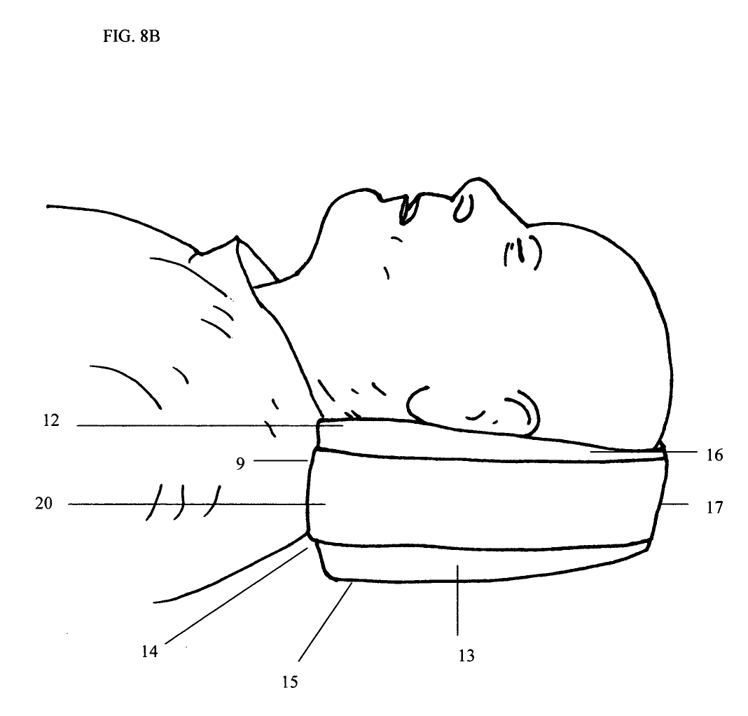

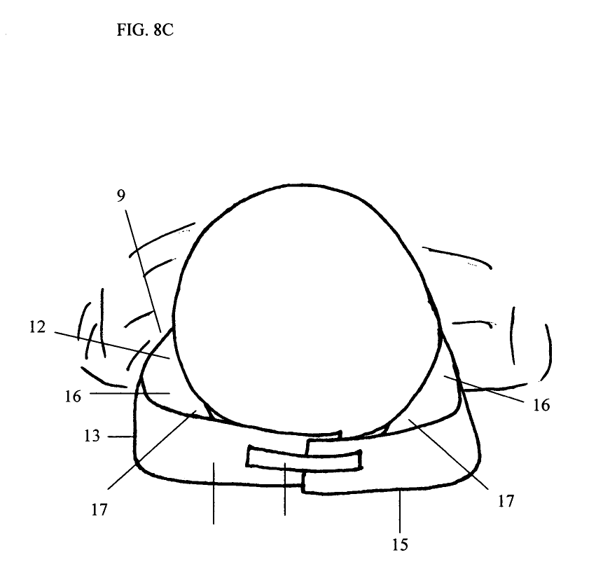

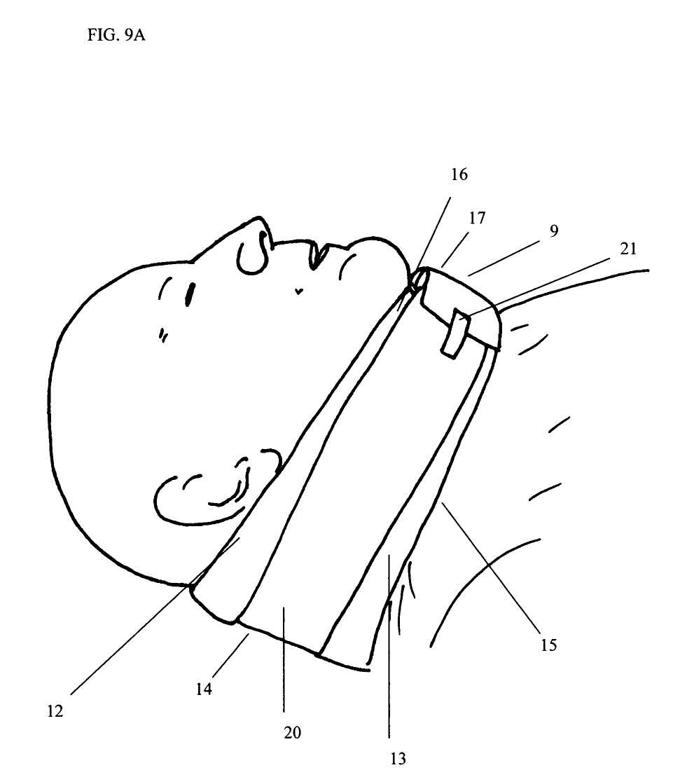

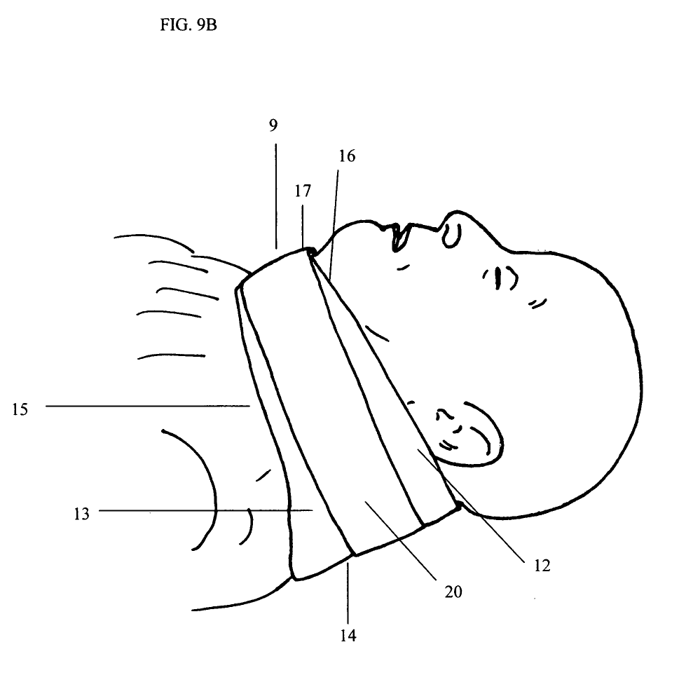

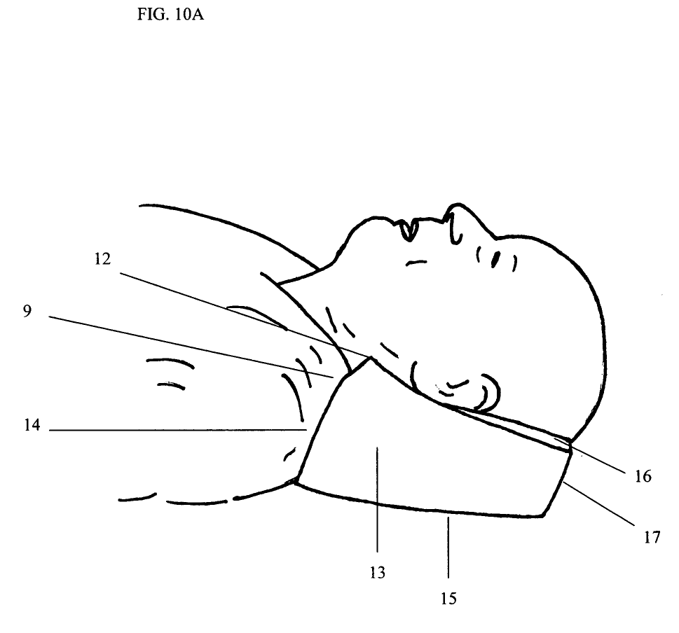

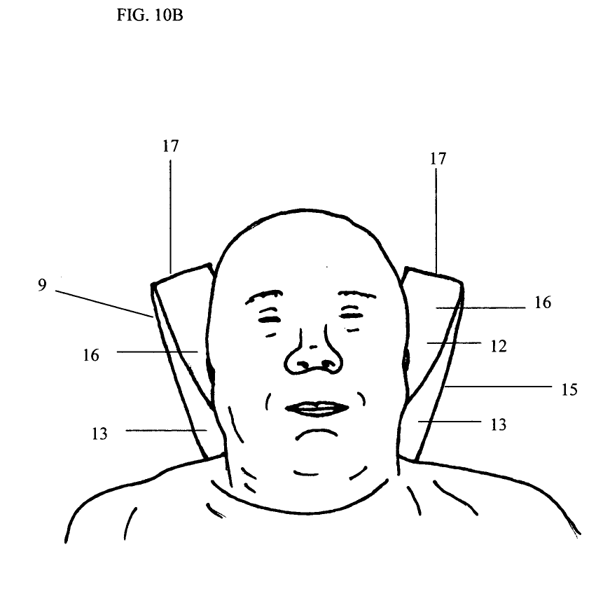

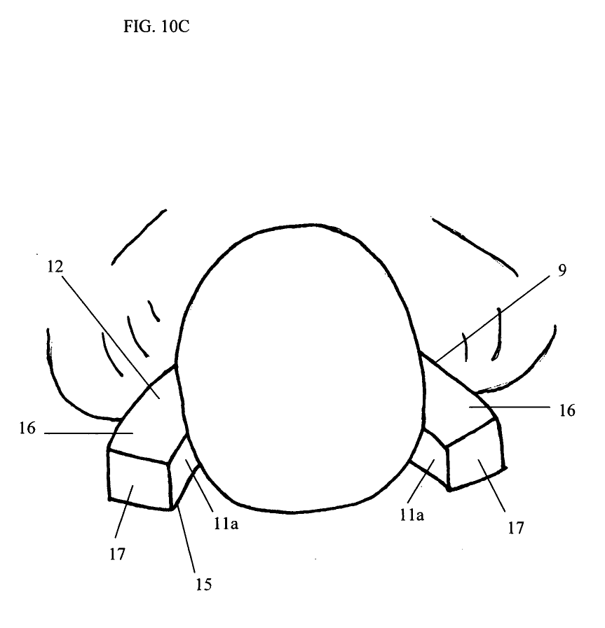

First, the support device 9 is positioned so that the thicker end 14 is behind the neck, as described by FIGS. 8A-C. Second, the support device 9 as shown in FIGS. 6A-C can be pushed directly behind the neck with the legs of the arch 16 extending generally upward and the exterior surface 14 resting on the bed. FIGS. 9A and B demonstrate this positioning. Third, the support device 9 can be placed behind the head with the thicker end 14 pushed under the neck and with the legs of the arch 16 extending on either side of the head, as described by FIGS. 10A-C. Each embodiment achieves pushing and holding the neck forward; elevating the chin thereby keeping the airway open; and stabilizing the head for surgery. Each embodiment is described below in further detail.

FIG. 7A is a right side view of the device 9 illustrating one of its positions during local anesthesia or monitored anesthesia care. With the patient in a supine position, the device 9 is between the chin and chest. For this usage, the support device 9 can originate in pillow position, as shown in FIGS. 6A-C, and is then rotated from around the back of the head to its position between the chin and chest. To rotate the device 9, strap 20 is unfastened and removed, the head is slightly lifted, and the device 9 is carefully turned about the neck while keeping its same orientation. If the device 9 has not been used as a pillow, then it can simply be placed into position between the chin and chest.

The neck trough 10, FIG. 2, fits over the front of the neck while interior sides 11a and 11b of neck trough 10, FIG. 2, contact the sides and front of the neck, respectively. The legs of the arch 16 point toward the bed with the base of each leg 17 near the bed. The top exterior surface 12 and bottom exterior surface 15 respectively contact the chin and chest. Labels 18, FIG. 3, and 19, FIG. 4, are removed to expose the adhesive strips which gently adhere to the chin and chest of the patient, securing the device in its position. The strap 20 is replaced and wrapped around the entire device, touching exterior surfaces 13, 14, and 17 and crossing the space created by the neck trough 10. The tightness of the strap is adjusted according to the size of the patient's head and length of the neck and then fastened with its tab 21, preferably at the thicker end 14.

FIGS. 7B and C are additional views of the device 9 placed between the chin and chest. FIG. 7B shows the device 9 placed between the chin and chest from the left side. FIG. 7C is a frontal view of this position.

FIG. 8A is a right side view of the device 9 stabilizing the airway, neck, and head from behind the neck and head. For this usage, the support device 9 can originate in pillow position, as shown in FIGS. 6A-C. It is then rotated 180 degrees behind the head so that the thicker end 14 is behind the neck. The strap 20 can remain wrapped around the device 9. If the device 9 has not been used as a pillow, then the head is lifted, and the device 9 is placed behind the head and neck with the thicker end 14 pushed under the neck. FIG. 8A also shows the strap 20 wrapped around the device 9 touching exterior surfaces 13, 14, and 17 and crossing the space created by the neck trough 10. Again, the tightness of the strap is adjusted according to the size of the patient's head and the length of the neck and fastened with its tab 21, preferably at the bases of the arch 17.

With the thicker end 14 of the device 9 pushed under the neck, the central portion 22, FIG. 1, of top exterior surface 12 contacts the back of the neck. Label 18, FIG. 3, marked “CHIN,” is not removed. The bottom exterior surface 15 contacts the bed, and label 19, FIG. 4, marked “CHEST” is also not removed. The neck trough 10, FIG. 2, and its legs 16 and their bases 17 now extend toward the top of the head. In this position, the head slightly tilts, causing the chin to elevate, and rests within the neck trough 10, FIG. 2.

FIGS. 8B and C are additional views of the device stabilizing the airway, neck, and head from behind the head. FIG. 8B shows the device 9 from the left side. FIG. 8C shows this position from the top of the head.

FIG. 9A represents a right side view of the device 9 in an alternate position used for stabilizing the airway, neck, and head from behind the head during local anesthesia or monitored anesthesia care. For this usage, the support device 9 can again originate in pillow position, as shown in FIGS. 6A-C. The strap 20 is then unfastened and removed, and the device 9 is pushed forward and generally upward until it supports the neck and contacts the patient's shoulders. If the device 9 has not been used in pillow position, then the head is lifted, the device 9 is placed behind the head and neck and then pushed forward and generally upward until it supports the neck and contacts the patient's shoulders.

The thicker exterior end 14 now touches the bed. Top exterior surface 12 contacts the lower portion of the back of the head and bottom exterior surface 15 contacts the patient's shoulders. Labels 18, FIG. 3, and 19, FIG. 4, are not removed. The legs 16 point generally upward, as positioned under the chin. The neck rests in the neck trough 10, FIG. 2, with the head slightly tilted and supported by the thicker end 14. The strap 20, as wrapped around the entire device, touches exterior surfaces 13, 14, and 17 and crosses the space created by the neck trough 10, FIG. 2, which now points toward the chin. The strap 20 is then fastened with tab 21, preferably at the bases of the legs of the arch 17, to secure the device in its position. FIG. 9B shows this second means of stabilizing the airway, neck, and head from behind the head from the left side.

FIG. 10A shows a left side view of the device 9 in the final proposed position for stabilizing the airway, neck, and head from behind the head. For this usage, the support device 9 can again originate in pillow position, as shown in FIGS. 6A-C. It is then rotated 180 degrees behind the head so that the thicker end 14 is behind the neck; the strap 20 is removed. If the device 9 has not been used as a pillow, then the head is lifted, and the device 9 is placed behind the head and neck with the thicker end 14 pushed under the neck.

With the thicker end 14 of the device 9 pushed under the neck, the central portion 22, FIG. 1, of top exterior surface 12 contacts the back of the neck. Label 18, FIG. 3, marked “CHIN,” is not removed. The bottom exterior surface 15 contacts the bed, and label 19, FIG. 4, marked “CHEST” is also not removed. The legs of the arch 16 point toward the top of the head and extend on either side of the head with the bases 17 near the top of the head. Exterior sides 13 face away from the patient while interior sides 11a contact the sides of the head. The strap 20 is not replaced for this position.

FIGS. 10B and C are additional views of the device stabilizing the airway, neck, and head from behind the head. FIG. 10B shows a frontal view of this position. FIG. 10C shows the device 9 from the top of the head.

During local anesthesia or monitored anesthesia care, the device 9 can be positioned according to FIGS. 7A-C, FIGS. 8A-C, FIGS. 9A and B, and FIGS. 10A-C to elevate the chin and stabilize the airway, neck, and head. Each of these placements, as proposed by the inventor, assists in preventing the chin from collapsing toward the neck and chest and consequently keeps the airway open and the head still. This enables the patient to breathe consistently without obstruction or stress and allows surgery to be completed in a safe manner.

What is claimed is:

1. An airway, neck, and head support used to help stabilize the airway, neck, and head of a patient comprising:
a device constructed of polyurethane foam having a carved-out central section adjoined by two lateral interior surfaces of equal length, height, and angle creating a neck trough, and surrounded by a continuous piece of foam forming top, bottom, and side exterior surfaces.

2. The airway, neck, and head support device of claim 1 wherein said neck trough is generally arch shaped.

3. The airway, neck, and head support device of claim 1 wherein said neck trough is accompanied by two vertical surfaces, or legs of the arch, extending at an equal length, height, and angle, to create the bases of the arch.

4. The airway, neck, and head support device of claim 1 wherein said neck trough and its surrounding, continuous piece of foam are additionally bordered by two lateral exterior surfaces of equal length, height, and angle adjoined by one horizontal exterior surface.

5. The airway, neck, and head support device of claim 1 wherein the width of the arch shaped space created by said neck trough and its legs are equal in width to the continuous, horizontal exterior surface at the opposing end.

6. The airway, neck, and head support device of claim 1 wherein said top exterior surface is marked “CHIN” and said bottom exterior surface is marked “CHEST” with removable labels covering corresponding adhesive strips.

7. The airway, neck, and head support device of claim 1 wherein said device is cut to selected dimensions and angles causing the end labeled “CHIN” and “CHEST” to be thicker than the opposing end.

8. The airway, neck, and head support device of claim 1 wherein said device is a continuous piece of polyurethane foam cut to its selected angles and dimensions by appropriate foam cutting saws.

9. The airway, neck, and head support device of claim 1 wherein said device is designed to fit a supine patient, given local or monitored anesthesia care, with the neck trough fitting over the front of the neck and the exterior top and bottom surfaces of the thicker end touching both the chin and chest, keeping the airway open by elevating the chin and stabilizing the head.

10. The airway, neck, and head support device of claim 9 wherein said device is further secured into position between the chin and chest with adhesive strips covered by removable labels of “CHIN” and “CHEST” and with an adjustable strap.

11. The airway, neck, and head support device of claim 10 wherein the strap used with said device is made of a flexible material, is long enough to extend completely around the device, and is secured, based on the size of the patient's head and length of the neck, with a tab.

12. The airway, neck, and head support device of claim 1 wherein said device can additionally be used by reclining or supine patients for purposes of providing comfort to the back of the neck and head, as a pillow, prior to the delivery of anesthesia.

13. The airway, neck, and head support device of claim 1 wherein said device is additionally designed to be placed in various selected positions behind the head to provide alternate means of keeping a supine patient's airway open, chin elevated, and head still during local or monitored anesthesia care.

14. The airway, neck, and head support device of claim 1 wherein said device is further designed to enable the user to rotate it around the patient's neck or behind the head for selected positions.
